(19) United States
(12) Patent Application Publication
(10) Pub. No.: US 2026/0118317 A1
(43) Pub. Date: Apr. 30, 2026

(54) SYSTEM INCLUDING GAS ANALYZER AND METHOD OF CONTROLLING THE SAME

(71) Applicant: ATONARP INC., Minato-ku, Tokyo (JP)

(72) Inventors: Hirofumi NAGAO, Tokyo (JP); Shinichi MIKI, Tokyo (JP); Kozue ASAKURA, Tokyo (JP)

(73) Assignee: ATONARP INC., Minato-ku, Tokyo (JP)

(21) Appl. No.: 19/162,070

(22) PCT Filed: Jun. 13, 2024
(86) PCT No.: PCT/JP2024/021439

§ 371 Date: Sep. 4, 2025

(30) Foreign Application Priority Data
Jun. 14, 2023 (JP) 2023-097953

(51) Int. Cl. G01N 27/66; G01N 33/00

(52) U.S. Cl. CPC G01N 27/66; G01N 33/0014; G01N 33/0073

(57) ABSTRACT

A system is provided, including an analyzer that analyzes components contained in a gas. The analyzer includes: a filter configured to pass components contained in the gas selectively; a detector configured to detect components that have passed through the filter; and a first control apparatus configured to set the filter so that the detector detects, as neon that is a main component included in the gas, an isotope 21Ne that has a low abundance ratio in place of an isotope 20Ne that has a high abundance ratio.

### TECHNICAL FIELD

The present invention relates to a system including a gas analyzer and a method of controlling the same.

### BACKGROUND ART

Japanese Laid-open Patent Publication No. 2006-145295 describes the provision of a real-time GC/MS trace gas detection and analysis system that captures and stores a trace gas sample, retains the sample for delivery to a GC column used for separation, separates the sample into sample components via the GC column, and analyzes all of the sample components in real time with a mass spectrometer. In this system, a gas flow distribution system captures a sample from a sample-bearing carrier gas in a micro-accumulator and sends the sample toward a gas chromatography column using a carrier gas which is supplied to the gas chromatography column to facilitate separation of the sample into sample components in real time, and transports the sample components to a mass spectrometer for detection and analysis or testing of trace gases.

Japanese Laid-open Patent Publication No. 2021-154240 discloses a rare gas (for example, krypton) recovery system capable of recovering and purifying rare gases from exhaust gas containing 100 ppm to 1% (preferably a maximum volume concentration of less than 500 ppm) of rare gas that is discharged from a semiconductor manufacturing apparatus. This system includes: an impurity removal unit that removes predetermined impurities from exhaust gas containing rare gas; a rare gas separation membrane module that separates into concentrated exhaust gas, which contains a high concentration rare gas (krypton), which has a higher rare gas concentration than the exhaust gas containing rare gas from which the predetermined impurities have been removed, and exhaust gas with a low rare gas concentration; and a rare gas adsorption unit that selectively adsorbs the rare gas from the concentrated exhaust gas.

Japanese Laid-open Patent Publication No. 2019-141752 discloses the provision of a laser gas recycling system and method of the same capable of obtaining purified gas, from which predetermined impurities have been removed, from exhaust gas discharged from an excimer laser oscillator apparatus. This laser gas recycling system is a laser gas recycling system for removing predetermined impurities from exhaust gas discharged from an excimer laser oscillator apparatus or the oscillation chamber of an excimer laser to obtain purified gas mainly composed of high-purity neon gas, and is equipped with a separation membrane apparatus that separates rare gases for excitation, which are not neon, from treated gas, and an impurity removal apparatus that removes the predetermined impurities from the treated gas that has permeated (or not permeated) through the separation membrane apparatus.

Japanese Laid-open Patent Publication No. 2005-123528 discloses a two-stage ArF excimer laser apparatus for exposure that operates at an oscillation frequency of 4 kHz or more and includes: an oscillation laser device including an oscillation chamber in which a first laser gas containing F₂ gas, Ar gas, and a first buffer gas is sealed; and an amplifier apparatus which includes an amplification chamber in which a second laser gas containing F₂ gas, Ar gas, and a second buffer gas is sealed, and amplifies and emits the laser beam emitted from the oscillation laser device, wherein the first buffer gas is He gas, or a mixture of He gas and Ne gas, and the second buffer gas is Ne gas.

### SUMMARY OF INVENTION

There are many processes in which gas containing a main component and trace components ranging from a few percent to the ppm order or even sub-ppm (ppb) order is used, and many applications in which the content of the trace components must be determined with high precision. One example is the field of gas lasers, which include known types such as helium-neon lasers, argon lasers, krypton lasers, carbon dioxide lasers, and excimer lasers. In particular, excimer lasers, which generate laser light using a mixture of rare gases, halogens, and the like, are widely used in fields such as machining, semiconductor manufacturing, and ophthalmic treatment. Not only for gas lasers, but for any process that uses gas, to improve product yield and precision, there is always demand to monitor the composition of gases being used in the process (including gas supplied to the process, gas generated by the process, gas discharged from the process, or gas produced as a by-product (hereinafter collectively referred to as “process gas”), and demand to manage (parameter management) equipment (processing equipment) that executes the process and/or the process itself based on the monitoring result.

In addition, from the viewpoint of resource conservation and the circular economy, there is an increasing demand to reuse gases that are discarded (exhausted) after use in processes and/or those that contain impurities. For such processing also, there is a strong demand for monitoring one or more compositions of the gas at each stage in a recovery and/or regeneration process. Conventional measurement apparatuses for measuring trace components in gases, such as GC/MS, are large, time-consuming, and require a long measurement time, typically using a carrier gas, such as helium. For this reason, there is demand for apparatuses and methods capable of analyzing trace components on-site with high accuracy using a simple mechanism.

One aspect of the present invention is a system including an analyzer that analyzes components contained in a gas. The analyzer includes: a filter that is configured to pass components contained in the gas selectively; a detector that is configured to detect components that have passed through the filter; and a first controller that is configured to set the filter so that the detector detects at least one of low-abundance isotopes of a first component, which have low abundance ratios in the first component, as the first component contained in the gas, in place of an high-abundance isotope of the first component, which has a high abundance ratio in the first component. The first controller may set the filter so that only one of the low-abundance isotopes of the first component is detected by the detector.

When the first component is the main component or one of the main components of the gas to be analyzed, the detection intensity will range from several times to several tens or several hundreds of times or higher than the detection intensity of one or more trace components, which makes it difficult to measure the first component and trace components with the same degree of accuracy. On the other hand, the abundance ratio of one or more less abundant isotopes (low-abundance isotopes, isotopes with low abundance ratios) of the first component may be a fraction, a few tenths, or a few hundredths, or even less of the abundance ratio of a more abundant isotope (high-abundance isotope, isotope with a high abundance ratio) of the first component. For this reason, in the past, isotopes with high abundance ratios were mainly (primarily) measured, while isotopes with low abundance ratios were either difficult to measure because they were buried in noise or were not given attention for measurement on their own.

In the present invention, as the first component, in place of a high-abundance isotope of the first component (an isotope with a high abundance ratio in the first component), that is, without measuring (detecting) the high-abundance isotope, at least any one of low-abundance isotopes of the first component (isotopes with a low abundance in the first component) or only one of the low-abundance isotopes is selected by the filter and measured (detected) by the detector. By doing so, it is possible to measure trace components, which are a fraction, a few tenths (less than several percent), or a few hundredths (less than one percent) or less of a main component, with the same or similar accuracy as the first component, which is a main (major, principal) component. This makes it possible for components contained in the gas, including trace components, to be measured with high accuracy, and for gas components to be analyzed with high precision. In addition, since there is no need to detect an extremely strong signal corresponding to the main component, deterioration of the detector can be suppressed.

The controller may include a second controller (second coordination function, second coordination apparatus, second coordination controller) that is configured to select, using the filter, a second component contained in the gas at a lower resolution than a resolution for selecting one of the low-abundance isotopes of the first component and detect using the detector. When the second component is present in even smaller amounts, the detection intensity for the second component can be increased by lowering the filter resolution for high intensity components. Accordingly, even when the content of the second component is smaller than the content of an isotope with a low abundance ratio of the first component, the second component, which is a trace component, and the first component, which is a main component, can be measured with the same or similar accuracy via the isotope with a low abundance ratio in the first component.

The analyzer may further include an ionizer that is configured to ionize components contained in the gas upstream of the filter. An ionization energy of the ionizer may be set to suppress ionization of the first component. This can suppress saturation of the ion source inside the analyzer caused by ionization of the first component that has a high abundance ratio, and suppress the influence of the first component on the measurement of other trace components including impurities. The analyzer may include a third controller that is configured to set the ionization energy of the ionizer so as to suppress ionization of the first component. One example of the ionizer includes a filament that emits thermal electrons.

The system may further include a chamber configured to temporarily hold gas upstream of the analyzer and an exhaust system that is configured to control the amount of gas flowing into the chamber. In addition, the exhaust system may be configured to keep a pressure P inside the chamber at a condition indicated below.

$$0.0 \; 05 Pa < P < 0.05 Pa \qquad ( 1 )$$

The lower limit of Condition (1) may be 0.008 Pa, and the upper limit may be 0.03 Pa.

The pressure of gas introduced into a mass spectrometer-type analyzer that filters by mass-to-charge ratio (m/z) is often controlled to an mPa (10⁻³ Pa) level or lower to suppress the influence of highly abundant components. In this system, the isotope of the first component that is most abundant (i.e., has the highest abundance ratio) is not measured, and one or more isotopes with a low or lower abundance ratio in the first component are measured. It is also possible to suppress ionization of the first component, which is the main component of the gas. This means that when the pressure in the chamber is increased, although the abundance of the first component, which is the main component, and one or more trace components in the chamber will increase, the measurement is less affected by the increase in the first component. For this reason, by increasing the pressure in the chamber, the abundance of the trace components will increase, which makes it possible to obtain an effect of increasing the measurement (detection) sensitivity for trace components.

One example of the first component is an inert gas, such as neon (Ne) or argon (Ar). Inert gases have high ionization energies. This means that it is easy to set an ionization energy that can suppress ionization of the main component but is less likely to obstruct ionization of the trace components. In addition, increasing the pressure inside the chamber only increases the amount of inert components, which lowers the risk of deterioration, such as oxidation of the filament, of the analyzer. The filter may include a quadrupole filter.

The system may further include: a processing apparatus into which or from which a process gas flows or is exhausted; a supply apparatus that is configured to supply at least one of an input gas, an intermediate gas, and an output gas as a process gas of the processing apparatus to the analyzer; and a management apparatus that is configured to manage the processing apparatus or a process being performed by the processing apparatus based on analysis results of the analyzer.

Another aspect of the present invention is a control method for a system including an analyzer for analyzing components contained in a gas. The analyzer includes a filter that is configured to selectively pass components contained in the gas and a detector that is configured to detect components that have passed through the filter, and the method includes detecting a first component contained in the gas using the detector with a controller of the analyzer selecting, using the filter, at least one of low-abundance isotopes of the first component, in place of a high-abundance isotope of the first component. The controller may control the analyzer to select, using the filter, only one of the low-abundance isotopes of the first component and detect with the detector. The method may include detecting a second component contained in the gas using the detector, with the controller selecting, using the filter, at a lower resolution.

The analyzer may further include an ionizer that is configured to ionize components contained in the gas upstream of the filter, and the method may further include controlling ionization energy by the controller to suppress ionization of the first component. The system may further include a chamber that is configured to temporarily hold gas upstream of the analyzer apparatus and an exhaust system that is configured to control an amount of gas flowing into the chamber, and the method may further include controlling by the controller to keep a pressure P inside the chamber at Condition (1) indicated above.

The system may further include: a processing apparatus into which or from which a process gas flows or is exhausted; and a supply apparatus that is configured to supply at least one of an input gas, an intermediate gas, and an output gas as the process gas of the processing apparatus to the analyzer, and the method may further include managing, by a management apparatus of the system, the processing apparatus or a process being performed by the processing apparatus based on analysis results of the analyzer.

Another aspect of the present invention is a method for analyzing components contained in a gas using an analyzer. The analyzer includes: a filter that is configured to pass components contained in the gas selectively; and a detector that is configured to detect components that have passed through the filter. The method includes detecting a first component contained in the gas using the detector with selecting at least one of low-abundance isotopes of the first component in place of a high-abundance isotope of the first component.

Another aspect of the present invention is a control program (or program product) of a system including an analyzer that analyzes components contained in a gas, which may be provided by being recorded on an appropriate recording medium. The analyzer includes a filter that is configured to pass components contained in the gas selectively and a detector that is configured to detect components that have passed through the filter, and the control program includes an instruction that causes a controller of the analyzer to detect a first component contained in the gas using the detector with selecting, using the filter, at least one of low-abundance isotopes of the first component in place of a high-abundance isotope of the first component.

### BRIEF DESCRIPTION OF DRAWINGS

FIG. 1 is a block diagram depicting an overview of a system including a gas analyzer.

FIG. 2 depicts example results of gas measurement.

FIG. 3 depicts how detection intensity changes according to ionization energy.

FIG. 4 depicts how detection intensity changes with chamber pressure.

FIG. 5 depicts example results of gas measurement.

FIG. 6 is a flowchart depicting an overview of system control.

### DESCRIPTION OF EMBODIMENTS

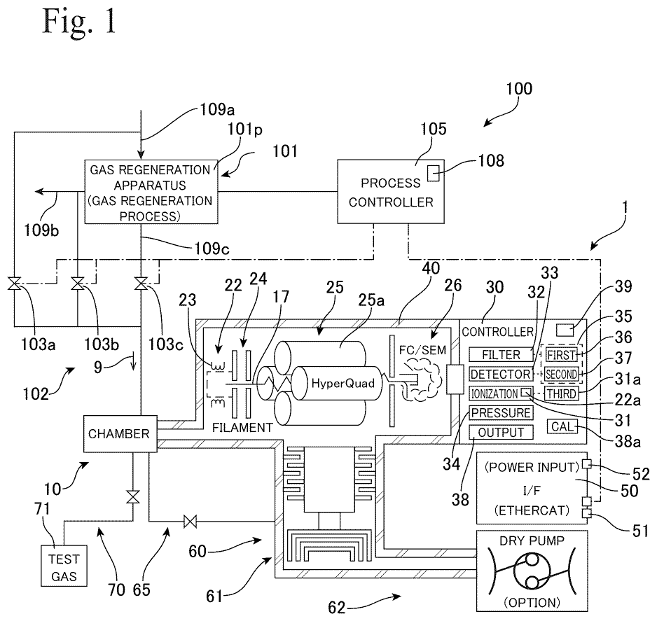

FIG. 1 depicts the schematic configuration of a process monitoring system 100 as one example of a system including a gas analyzer (gas analyzer apparatus, gas analyzer device) 1. The system 100 shown as an example is a system that manages a gas regeneration apparatus (recirculation apparatus, gas processing apparatus) 101 and a gas regeneration process 101p executed by the gas regeneration apparatus 101. The system 100 includes a supply apparatus 102 that is configured to supply, as process gas, gas 109a supplied to the process 101p of the gas regeneration apparatus 101, gas 109b that is processed and outputted by the process 101p, and gas 109c that is being processed in the process 101p, individually by switching between them or in parallel to the gas analyzer 1 for a gas to be analyzed (sample gas) 9, and a management apparatus (process controller) 105 that is configured to manage the gas regeneration apparatus 101 and/or the process 101p by referring to analysis results of the gas analyzer 1. The supply apparatus 102 according to the present embodiment includes switching valves 103a, 103b, and 103c that switch between gases 109a, 109b, and 109c, and supply these gases to the gas analyzer 1.

The processing apparatus that performs a process into which or out of which the process gas 109 to be analyzed by the gas analyzer 1 flows is not limited to the gas regeneration apparatus 101. The processing apparatus may be an apparatus that performs a process including, as examples, CVD (Chemical Vapor Deposition) or PVD (Physical Vapor Deposition) in a semiconductor process, for forming various types of film or layer on a substrate or for etching a substrate. The processing apparatus is not limited to processes related to semiconductor manufacturing, and may be an apparatus that performs processes for laminating various types of thin films on optical components, such as lenses and filters, as substrates, an apparatus that blends, regenerates, and recovers gases used in semiconductor manufacturing, or a processing apparatus that handles gases for other purposes.

One example of the gas regeneration apparatus 101 is disclosed in Japanese Laid-open Patent Publication No. 2019-141752 mentioned earlier. One example of the gas regeneration apparatus 101 is an apparatus (laser gas recycling system) that obtains a purified gas, from which predetermined impurities have been removed, from exhaust gas discharged from an excimer laser oscillator apparatus. One example of the exhaust gas (process gas) 109a to be treated or processed is described as containing neon as the main component, rare gases (krypton, xenon, argon) at 1 to 10% and preferably 1 to 8% of the total mass, and, as examples of the impurities in the exhaust gas, CF4 (CF₄), N2 (N₂), and He, where the CF₄ concentration in the exhaust gas is expected to be in a range of 1 ppm to 500 ppm.

As an example of a laser gas whose main component is neon, Japanese Laid-open Patent Publication No. 2005-123528 discloses, as one example of ArF gas, a mixed gas containing 3.5% argon (Ar) and 10 ppm (0.001%) xenon (Xe), with neon (Ne), that is, 96.499%, as the remainder. Accordingly, when monitoring the components of a gas being processed and a gas being recycled in an apparatus 101 that handles such gas and is being monitored, it is desirable for the gas analyzer 1 to monitor trace or impurity components that exist in concentrations (mixing ratios) of the order of % to ppm or lower relative to the main component quantitatively, on-site, and in real time.

The process monitoring system 100 that uses the gas analyzer 1 in the present embodiment performs real-time monitoring of the gas 9 to be analyzed, and by providing highly reliable measurement results (analysis results) enables the provision of innovative process control. As one example, the gas analyzer 1 can monitor in real time the gas components used in each process or step involved in semiconductor manufacturing, and functions as a total solution platform aimed at dramatically improving throughput in semiconductor chip manufacturing and maximizing the yield rate. In addition, when combined with a system for gas generation, recovery, and regeneration, the gas analyzer 1 also serves as a platform for reviewing processes and maximizing the utilization efficiency of resources from viewpoints such as resource conservation and the circular economy.

The gas analyzer (analyzer apparatus) 1 according to the present embodiment is typically an extremely small-scale mass spectrometer (spectrometer apparatus) that can be directly connected to or incorporated (embedded, built) into an apparatus to be analyzed. The gas analyzer 1 can be equipped with standard protocols that are primarily used in semiconductor manufacturing process apparatuses, such as the EtherCat (registered trademark) protocol 51, and can be integrated into a processing equipment control system, such as the process monitoring system 100.

The gas analyzer 1 is an apparatus for analyzing components contained in the gas (sample gas, sampling gas) 9. The gas analyzer 1 includes a chamber 10 configured to temporarily hold the sample gas 9 supplied from a process side by the supply apparatus 102, an ionizer (ionizer apparatus) 22 configured to generate ions (an ion flow) 17 of the sample gas 9, a filter 25 configured to selectively pass components contained in the gas, and a detector 26 configured to detect the components that have passed through the filter. One example of the gas analyzer 1 is a mass spectrometry type detector (mass spectrometer (MS)). The filter 25 may include a filter unit (typically a mass filter, in the present embodiment, a quadrupole filter) 25 that filters (or selects, sorts, or turns on and off) a sample gas (that is, sample gas ions) 17, which is ionized and has been supplied from the ionizer 22, according to mass-to-charge ratio. The detector 26 may be a detector that detects filtered ions, which is to say, detects ion intensity or an ion current.

The gas analyzer apparatus 1 further includes a vacuum vessel (housing) 40 that houses the filter unit 25 and the detector 26, and an exhaust system 60 that can keep (maintain) the inside of the housing 40 and the chamber 10 connected to the housing 40 at an appropriate negative pressure condition (vacuum condition). Although the chamber 10 is important as a location for controlling the conditions (pressure) of the sample gas 9 flowing into the gas analyzer apparatus 1, to enable real-time measurement, the chamber 10 may have a minimum capacity, as examples, a vessel or buffer with a capacity of 1 to several tens of cm³ or 1 to several cm³, and may be constructed or configured from part of a pipe that also serves as the supply apparatus 102.

The exhaust system 60 in the present embodiment includes a turbomolecular pump (TMP) 61 and a roots pump (or dry pump) 62, and controls the internal pressure of the chamber 10 via the housing 40 in which the filter 25 and the detector 26 are incorporated. The dry pump 62 may be provided as an option. Another type of pump may be used in the exhaust system 60, which may be a single-stage exhaust system or a multi-stage exhaust system with three or more stages. The exhaust system 60 may include an exhaust path 65 that bypasses the housing 40 from the sample chamber 10, and may direct the sample gas 9 to the sample chamber 10 and control the internal pressure of the sample chamber 10 separately from the gas flow rate used for filtering. When analyzing gas that has been discarded or discharged, a large amount of sample gas 9 can be drawn from the process into the sample chamber 10 relative to the amount of gas (that is, the amount of ions) supplied to the filter 25, which makes it possible to provide a gas analyzer 1 that can monitor the state (fluctuations) of the process 101p in real time.

One example of the filter 25 is a mass filter, which includes four cylindrical or columnar electrodes (HyperQuads) 25a whose insides (interiors) are finished to form hyperbolic surfaces, thereby creating a hyperbolic electric field for filtering according to mass-to-charge ratio. Such a quadrupole-type mass filter 25 may be configured by disposing a large number of, for example, nine, cylindrical electrodes in a matrix (array) so as to form a plurality of pseudo-hyperbolic electric fields. A Faraday Cup (FC) and a secondary electron multiplier (SEM) can be given as examples of the detector 26. The detector 26 may use such devices in combination or may switch between them. The detector 26 may be of a different type, such as a Channel Electron Multiplier (CEM) or a Microchannel Plate (MP).

The ionizer (ionizer apparatus) 22 includes an electron ionizer (filament or EI ion source) 23 that ionizes (that is, electronically ionizes) the sample gas 9 supplied from the process 101p via the gas supply apparatus 102 and the chamber 10 by electron impact (thermal electrons). Although the EI ion source 23 is usually operated at a high vacuum of 10⁻³ Pa or lower, in the present embodiment, when the main component of the sample gas 9 is an inert gas, it is possible for the EI ion source 23 to operate at a low vacuum of 10⁻² Pa or higher. The gas analyzer 1 may include one or a plurality of lenses (ion lenses, electrostatic ion lenses) 24 that are configured to direct the ionized gas 9 as an ion flow (ion beam) 17 to the filter 25.

The gas analyzer 1 includes a controller (control box, control module) 30 that controls each module in the analyzer 1, and an interface apparatus 50 for communication with the outside. The controller 30 includes computer resources, such as a CPU and memory, and controls the gas analyzer 1 by loading and executing a program (or program product) 39. The program 39 can be provided by being recorded on a computer-readable medium. The interface (interface apparatus) 50 includes a power input interface 52 and a communication interface 51 that conforms to EtherCat.

The control module (controller) 30 may have a function (ionization controller) 31 for controlling the ionizer apparatus 22, a function (filter controller) 32 for controlling the mass filter 25, a function (detector controller) 33 for detecting the ions (an ion current) that have arrived via the detector 26, and a function (pressure controller) 34 for controlling the exhaust system 60 to control the pressure in the chamber 10. These controllers 31 to 34 may have a function of controlling each of the controlled (control target) apparatuses (units) so as to maintain set values or conditions. The controller 30 may also have a function (cooperative control apparatus) 35 of performing predetermined measurements by cooperatively controlling a plurality of apparatuses (units) of the gas analyzer 1. The cooperative control apparatus may include, for example, a function where the controller 30 sets a predetermined condition in the filter 25 and detects components selected by that condition with the detector 26. The ionization controller 31 that controls the ionizer 22 may include a function as an ionization energy control apparatus (filament control apparatus) that controls the filament current and/or voltage supplied to the filament 23.

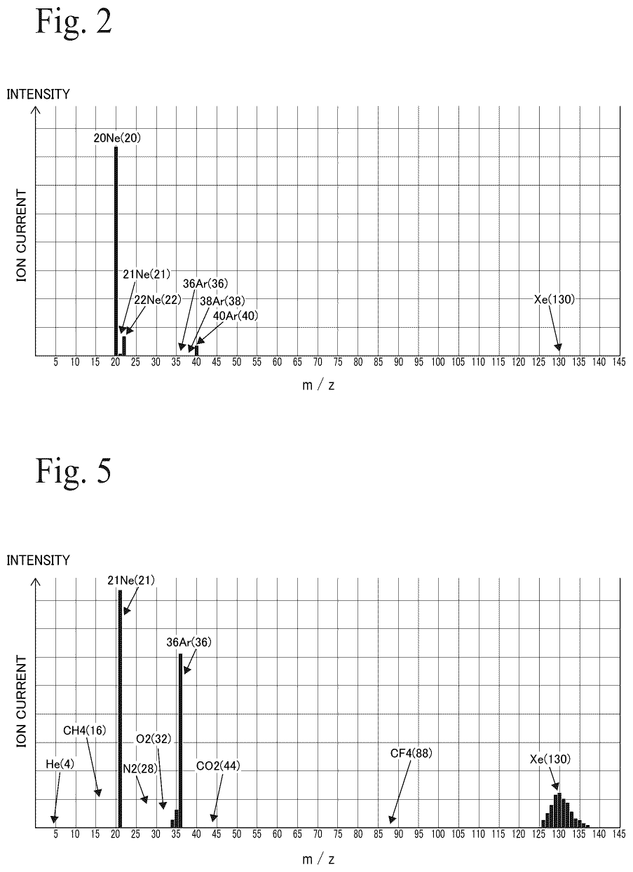

FIG. 2 depicts an example where the process gas (first mixed gas) 109b regenerated by the gas regeneration process 101p of the gas regeneration apparatus 101 described above is measured as the sample gas 9. This first mixed gas 109b is an ArF gas, and as one example, it contains 3.5% of argon (Ar), 10 ppm (0.001%) of xenon (Xe), and 96.499% of neon (Ne). FIG. 2 shows example results obtained when the first mixed gas 109b is used as a sample gas and measured by a conventional mass spectrometer (MS). The results of this mass spectrometry are obtained as intensities for different mass-to-charge ratios (m/z).

Along with helium and krypton, chemically stable inert gases include neon, argon, and xenon. Neon is the element with the atomic number 10, and its stable isotopes include 20Ne with a mass-to-charge ratio of 20 which accounts for 90.48%, 21Ne with a mass-to-charge ratio of 21 which accounts for 0.27%, and 22Ne with a mass-to-charge ratio of 22 which accounts for 9.25%. Argon is the element with the atomic number 18, and its stable isotopes include 40Ar with a mass-to-charge ratio of 40, which accounts for 99.6%, 38Ar with a mass-to-charge ratio of 38, which accounts for 0.063%, and 36Ar with a mass-to-charge ratio of 36, which accounts for 0.337%. Xenon is the element with the atomic number 54, and its stable isotopes include 132Xe with a mass-to-charge ratio of 132 which accounts for 26.9%, 131Xe with a mass-to-charge ratio of 131 which accounts for 21.2%, 130Xe with a mass-to-charge ratio of 130 which accounts for 4.07%, 129Xe with a mass-to-charge ratio of 129 which accounts for 26.4%, 128Xe with a mass-to-charge ratio of 128 which accounts for 1.91%, and 126Xe with a mass-to-charge ratio of 126 which accounts for 0.089%.

If this mixed gas 109b were measured using a conventional mass spectrometer, neon would compose more than 96% of the components in mixed gas 109b, and the concentration (content) of xenon is approximately six orders of magnitude (10⁻⁶) lower than the concentration of neon. This means that it would be difficult to simultaneously detect neon and xenon using the same detector under the same conditions. In particular, it is difficult to measure each component simultaneously with high accuracy, and it is also difficult to maintain the working life of the detector due to the huge ion current when neon is measured. In addition, even if attempts were made to measure argon at the same time, it is believed that the ionization of neon, which comprises the majority of the gas, would saturate the inside of the ionizer and inhibit the ionization of argon. Accordingly, it was not thought possible to measure this type of sample gas 9 using a mass spectrometry-type gas analyzer (analysis) apparatus.

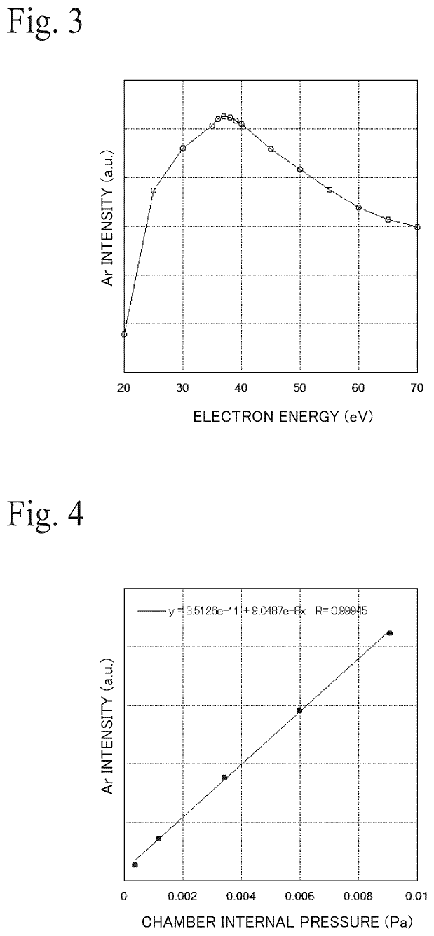

FIG. 3 depicts one example of the argon intensity obtained when the pressure inside the chamber 10 is set at 10⁻² Pa and the mixed gas 109b is measured while varying the ionization energy (i.e., the thermal electron energy) of the ionizer 22. In a conventional mass spectrometer, the ionization energy of the ionizer apparatus is set at 70 eV because mass spectra registered in existing databases, such as the NIST database, were measured at 70 eV. However, it can be understood that in this case, although the intensity of argon ions will increase as the ionization energy is increased, this increasing trend peaks at an ionization energy of approximately 37 eV, and the intensity of argon thereafter decreases as the ionization energy is increased toward 70 eV. Accordingly, it is believed that when the ionization energy exceeds approximately 37 eV, the inside of the ionizer apparatus 22 will become saturated with ionized neon, which comprises the majority of the mixed gas 109b, so that the ionization of argon is inhibited. For this reason, it is understood that for a gas like the mixed gas 109b in which the main component (major element, principal component, first component) comprises several tens of percent, suppressing the ionization of the main component can promote ionization of other components.

FIG. 4 depicts the relationship between the pressure in the chamber 10 and the measured intensity of argon when the ionization energy was set at 35 eV during measurement of the mixed gas 109b. Increasing the pressure inside the chamber increases the amount of argon introduced into the filter 25, which should raise the intensity measured by the detector 26. From these measurement results, it was verified that even when the ionization energy was set lower than the value set in a conventional mass spectrometer, the measured intensity (detected intensity) of argon increased almost in proportion to the pressure in the chamber.

These inert gas components have relatively high ionization energies, with neon having an ionization energy of approximately 21.6 eV, argon having an ionization energy of approximately 15.8 eV, and xenon having an ionization energy of approximately 12.1 eV. Accordingly, for the ionizer 22, although ionization at an energy level that exceeds the ionization energy of the respective components has the advantage of increasing the amount of ions, there can also be an adverse effect if the amount of ions increases excessively. In particular, when, like the mixed gas 109b in the present embodiment, the ionization energy of the main component (first component) in the gas is high, by lowering the ionization energy in the ionizer 22 to a predetermined range, it should be possible to find an ionization energy condition that suppresses the ionization of the main component and has almost no effect on the ionization of the trace components. As one example, the target value of the ionization energy of the ionizer 22 can be set at a value that is insufficient or marginal (that is, a value that causes ionization but is not high enough to ionize all molecules) as the ionization energy of the main component and is sufficiently higher than the ionization energy of the trace components.

The ionization control apparatus (ionization controller) 31 of the controller 30 of the gas analyzer 1, according to the present embodiment, may include a function for maintaining the ionization energy of the ionizer 22 at a set value 22a that satisfies the above condition. The controller 30 may include a third control apparatus (third controller) 31a that sets the ionization energy of the ionizer 22 to a value 22a that suppresses ionization of the first component. When the mixed gas 109b described above is analyzed as the sample gas 9, the third controller 31a may set the set value 22a of the ionization energy at 35 eV or at around that value.

The cooperative control function 35 of the controller 30 includes a first control apparatus (first controller) 36 configured to set the filter 25 so that the detector 26 detects, as a first component contained in the gas (sample gas) 9 to be measured, at least one of the low-abundance isotopes of the first component that have a low abundance ratio in the first component instead of high-abundance isotope of the first component that has a high abundance ratio in the first component. The first controller 36 includes a first coordination function (first coordination control apparatus) that is configured to select at least one of the low-abundance isotopes of the first component using the filter 25 and detect such isotopes with the detector 26. The first controller 36 may be configured to set the filter 25 so that only one (any one) of the low-abundance isotopes (single low-abundance isotope) of the first component is detected by the detector 26.

The controller 30 may further include a second control apparatus (second controller) 37 that is configured to set the filter 25 so that the detector 26 detects a second component contained in a trace amount in the sample gas 9 with a lower resolution than that required for selecting a low-abundance isotope of the first component. The second controller 37 includes a second coordination function (second coordination control apparatus) that is configured to select the second component with a trace amount using the filter 25 with low resolution and detect the second component using the detector 26.

The controller 30 may include a pressure control apparatus (pressure controller) 34 that is configured to control the exhaust system 60 to keep the pressure P inside the chamber 10 at the following condition.

$$0.005 Pa < P < 0.05 Pa \qquad ( 1 )$$

The lower limit of Condition (1) may be 0.008, and the upper limit may be 0.03.

FIG. 5 depicts the results of a simulation in which the ArF gas (first mixed gas) 109b described earlier is measured as the sample gas 9 by the gas analyzer 1 according to the present embodiment. First, the first controller 36 sets the filter 25 so that as the first component (neon), which is the main component in the sample gas 9, in place of the isotope (20Ne) with a high abundance ratio in the first component, that is, without measuring (detecting) a high-abundance isotope (20Ne) of the first component, at least any one of the low-abundance isotopes of the first component, in this example 21Ne only, is selected, and has the detector 26 measure (detect) the ion flow at the time that 21Ne has passed through the filter 25. The first controller 36 may set the filter 25 so that 20Ne does not pass through (that is, no interval where 20Ne passes through is set), and/or the detector 26 may be turned off when 20Ne passes through the filter 25.

In the gas analyzer 1, the first controller 36 causes the filter 25 to select 21Ne, which has an intensity of around 0.27% (10⁻⁴) in neon, in place of 20Ne so that the detector 26 can detect 21Ne only as neon. As a result, neon, the main component of the sample gas 9 and accounting for over 90% of the total, can be detected at an intensity comparable to that of a trace component present in the parts per million (ppm) range. Accordingly, in the gas analyzer apparatus 1, the components contained in the gas 9, including the main component as well as the trace components, can be measured with high accuracy, even for trace amounts. In addition, with the gas analyzer apparatus 1, since there is no need to detect a very high intensity signal corresponding to the main component, it is possible to suppress deterioration of the detector 26.

In the gas analyzer 1 according to the present embodiment, even for the case of argon, which has the second highest mixing ratio in the ArF gas 109b, the first controller 36 may use the filter 25 to select only the isotope 36Ar, which has a low abundance ratio in argon, instead of the isotope 40Ar, which has a high abundance ratio, and detect only the 36Ar with the detector 26. Accordingly, in the gas analyzer 1, argon, which has the second highest abundance ratio after neon in the sample gas 9, can be detected with the same or similar intensity as other trace components.

In addition, in the gas analyzer 1, using the pressure controller 34, the pressure P inside the chamber 10 can be set one order of magnitude or higher than a conventional filament-type mass spectrometer apparatus, thereby increasing the amount of sample gas 9 flowing into the gas analyzer 1. On the other hand, the ionization energy control function 31a of the ionization controller 31 lowers the set value 22a of the ionization energy to 35 eV to suppress ionization of isotopes of neon, which is the main component of the sample gas 9. As a result, the amount of ions of trace components aside from neon can be relatively increased, which improves detection sensitivity for the trace components. Note that since the main component of the mixed gas (ArF) 109b to be measured is the inert gas neon, increasing the inflow rate is expected to have hardly any effect on the lifespan of the filament 23. Impurities may also originate from the chamber 10 or the supply apparatus 102 that supplies gas to the chamber 10, or from a similar source. However, by increasing the amount of gas 9 flowing into the chamber 10, it is possible to increase the amount of impurities originating from the gas regeneration apparatus 101 or the process 101p, which has the advantage of improving the detection sensitivity for trace components that are factors in controlling the apparatus 101 or the process 101p.

In the gas analyzer 1, the second controller 37 selects a second component (xenon) present in a trace amount in the sample gas 9 from the process gas (ArF gas) 109b using the filter 25 with a lower resolution than the resolution for selecting the isotope (21Ne) that is the low-abundance isotope of the first component (neon), and detects this second component with the detector 26. The second controller 37 controls the scan units (scan interval, resolution of mass-to-charge ratio m/z) of scanning in the gas analyzer 1, and when measuring neon, controls the filter 25 and the detector 26 so as to detect only the isotope 21Ne with the resolution of 1 AMU or higher, for example. On the other hand, when measuring xenon, the second controller 37 controls the filter 25 and the detector 26 to detect a wide range (low resolution) which includes a plurality of isotopes of xenon. When measuring xenon, the second controller 37 may also control the filter 25 to detect with a resolution of 1 AMU or higher, and output an aggregate (i.e., an integral) of measurement results of the xenon isotopes from the detector 26 as the xenon detection result. The second controller 37 may set the filter 25 to detect every stable isotope of xenon, that is, 132Xe, 131Xe, 130Xe, 129Xe, 128Xe, and 126Xe, as the intensity of xenon, and detect such isotopes using the detector 26.

With this control and settings, the gas analyzer 1 is able to detect xenon, which is present in the first mixed gas (ArF gas) 109b in the order of ppm, at an intensity that is substantially equal to or close to neon, which composes nearly 100% of the gas, and argon, which composes several percent. This means that the gas analyzer 1 can measure, analyze, and monitor trace components in the process gas 109b in real time simultaneously with the main component (major element). In the same way, it can be understood that even if the process gas 109 contains impurities such as helium (He), methane (CH4), nitrogen (N2), oxygen (O2), carbon dioxide (CO2), and carbon tetrafluoride (CF4) at the ppm or sub-ppm (that is, ppb) level in addition to the intended components of the mixed gas (ArF gas), such impurities can be measured with high accuracy in real time and the measurement result can be used to manage the process 101p.

In addition, the system 100 may include a calibration apparatus 70 that is configured to measure a reference gas (test gas) 71 containing known concentrations in advance by the gas analyzer 1 with the measurement conditions set as described above to calibrate the detection intensity of trace components, such as argon and xenon, and the detection intensity of neon, which is the main component. The controller (control module) 30 of the gas analyzer 1 may be equipped with a mixing ratio output apparatus (arithmetic function, computation apparatus, or output apparatus) 38 that stores a calibration result 38a and, when measuring the sample gas 9 on-site, acquires (calculates) the mixing ratios of trace components based on the calibration result 38a from the ratios of the detection intensities of other components to the detection intensity of neon, which is the main component (first component). Using the gas analyzer 1, it is possible to accurately determine trace components or impurities on-site. Accordingly, it is possible to use measurement results (analysis results) of the gas analyzer 1 to control, manage, and monitor the process 101p. The function of determining the mixing ratio may be implemented in an external apparatus, such as the process controller 105.

The gas to be measured by the gas analyzer 1 may be another gas, for example, KrF gas. As a gas for a xenon laser, gas containing krypton (KrF gas) in place of or in addition to xenon is known. Krypton (Kr) is the element with the atomic number 36, and is composed of stable isotopes including 86Kr with a mass-to-charge ratio of 86 which accounts for 17.3%, 84Kr with a mass-to-charge ratio of 84 which accounts for 57%, 83Kr with a mass-to-charge ratio of 83 which accounts for 11.5%, 82Kr with a mass-to-charge ratio of 82 which accounts for 11.6%, 80Kr with a mass-to-charge ratio of 80 which accounts for 2.25%, and 78Kr with a mass-to-charge ratio of 78 which accounts for 0.35%. Accordingly, even in a system 100 that measures a mixed gas containing ppm to several percent of Kr, the gas analyzer 1 according to the present embodiment can accurately detect and analyze Kr, including trace amounts of impurities, in the same way as a mixed gas containing xenon.

The analysis results of the gas analyzer 1 can be supplied via a communication interface 51, such as the EtherCat of the interface apparatus 50, to the process controller 105. The analysis results may also be provided via the cloud to other external apparatuses that monitor the process 101p. The process controller 105 may include computer resources, such as a CPU and memory, and may be operated by a control program (program product) 108. One example of the process 101p controlled and/or monitored by the process controller 105 is a process executed in a processing apparatus 101 into which a gas used in a semiconductor process flows or is discharged, where at least one of input, intermediate, and output process gases 109a to 109c of the processing apparatus 101 is supplied via the supply apparatus 102 to the gas analyzer 1 and analyzed, which makes it possible to control and monitor the processing apparatus 101 and the process 101p based on the results.

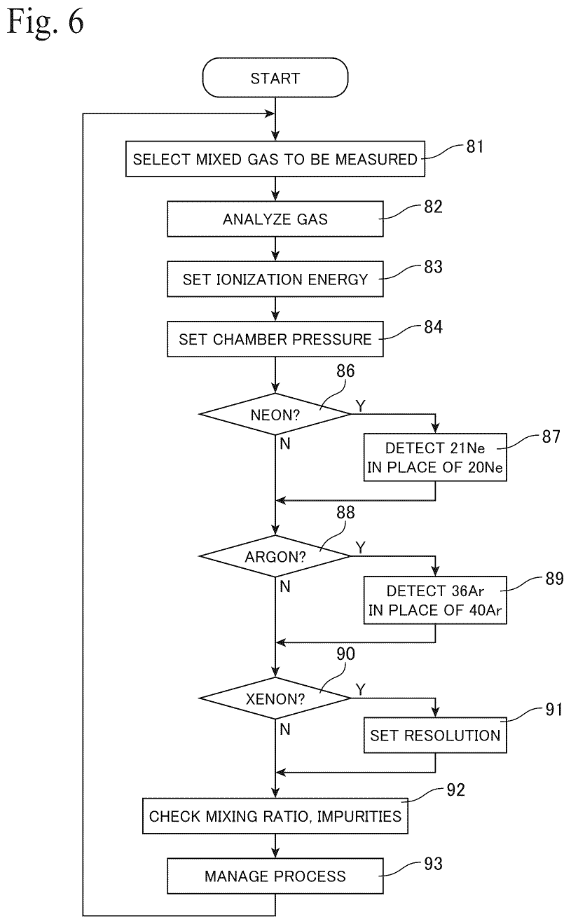

FIG. 6 depicts one example of a control process (control method, control program) in the process monitoring system 100 including the gas analyzer 1. In step 81, at least one of the input gas 109a, the intermediate gas 109b, and the output gas 109c of the processing apparatus 101, into which a process gas flows or is discharged, is selected by the supply apparatus 102 and supplied to the gas analyzer 1 as the sample gas 9. In step 82, the gas analyzer 1 starts gas analysis. First, in step 83, in the gas analyzer 1, the ionization control apparatus (third controller) 31a sets the ionization energy 22a of the ionizer 22 at a suitable value for the gas 9 to be measured. In this example, when measuring a mixed gas containing neon as the main component (first component), as one example 35 eV is set as the ionization energy 22a capable of suppressing the ionization of neon. Next, in step 84, in the gas analyzer apparatus 1, the pressure controller 34 sets the pressure of the chamber 10 at the range of Condition (1), which is higher than conventional conditions and thereby increases the amount of trace components supplied to the gas analyzer apparatus 1.

In step 86, when measuring neon, which is the main component (first component) with the highest content ratio, in step 87, the first controller 36 uses the filter 25 to select 21Ne, which is an isotope with a low abundance ratio, in place of 20Ne, an isotope with a high abundance ratio so that 21Ne is detected by the detector 26 (without 20Ne being measured or detected). Although it is possible to select and detect a plurality of low-abundance isotopes, when the main component is detected with an intensity that is similar to a trace component, especially an extreme trace component of the order of ppm, it is preferable for only one of the isotopes with a low abundance ratio to be selected using the filter 25 and detected by the detector 26 (that is, to limit detection to any one of isotopes).

In step 88, when the next most content ratio component argon is to be measured, in step 89, the first controller 36 uses the filter 25 to select the isotope 36Ar with a lower abundance ratio in place of the isotope 40Ar with a higher abundance ratio and detects 36Ar with the detector 26 (without 40Ar being measured or detected).

In step 90, when measuring the trace component xenon (the second component), in step 91, the second controller 37 selects xenon isotopes using the filter 25 with a lower resolution than the resolution for selecting 21Ne and/or 36Ar, which is an isotope with a low abundance ratio in the major component, and detects the selected isotopes with the detector 26. As one example, all of the xenon isotopes 126Xe, 128Xe, 129Xe, 130Xe, 131Xe, and 132Xe are selected using the filter 25 and are detected with the detector 26, and the sum (integral value) of these measurements is acquired as the measured value of xenon.

In step 92, in the gas analyzer 1, the mixing ratio output apparatus 38 may output a mixing ratio based on the calibration result 38a to the process controller 105. In addition, information of impurities at the ppm or sub-ppm level, such as helium (He), methane (CH4), nitrogen (N2), oxygen (O2), carbon dioxide (CO2), and carbon tetrafluoride (CF4), obtained during the gas analysis process, may be output to the process controller 105. In step 93, the process controller 105, as a process management apparatus, manages the processing apparatus (gas regeneration apparatus) 101 or the process (gas regeneration process) 101p performed by a processing apparatus based on the measurement results (analysis results) of the gas analyzer 1.

As described above, this embodiment of the present invention provides a method that uses the gas analyzer 1 to perform rapid analysis of impurities in Ne gas used in an excimer laser. It is necessary for Ne gas for an excimer laser to have concentrations of Ar, Xe, Kr, and the like within certain ranges. In addition, it is necessary to ensure that components that are unsuitable for lasers, such as N2, O2, and CO, are kept below certain concentrations. When considering factors such as analysis time, space, and carrier gas consumption, it is also desirable to input Ne gas directly into a mass spectrometer and measure impurities in the Ne gas in real time without using a gas chromatograph (GC) or the like.

For this reason, it is desirable to improve the analytical sensitivity of Xe and the impurities (as examples, N2, O2, CO, and CO2 in Ne) in Ne gas. In the gas analyzer 1, the pressure controller 34 enables a larger amount of gas (for example, about 10⁻² Pa) to be introduced into the mass spectrometry chamber 10 than usual (for example, an upper limit of 10⁻³ Pa). This enables a larger amount of impurities to be introduced into the mass spectrometry chamber.

In particular, impurities such as N2 and O2 are always present in certain amounts as background components in a mass spectrometry chamber, in addition to any impurities originating from Ne gas. This means that it is necessary to distinguish whether such impurities originate from the chamber or from the Ne gas. By introducing as much Ne gas as possible into a mass spectrometry chamber and increasing the amount of impurities originating from the Ne gas relative to the background impurities present in the vacuum, it becomes possible to measure the concentrations of impurities with higher sensitivity. Also, since Ne gas is an inert gas, even if a larger amount of the gas than usual (normal case) is introduced into the mass spectrometry chamber 10, there will be minimal damage to the filament 23.

Next, the ionization energy is controlled by the ionization energy control apparatus (third controller) 31a. If a large amount of Ne gas were simply introduced into the mass spectrometry chamber 10, the ion source would be saturated with Ne ions, which would conversely have a negative effect on the analytical accuracy of concentrations of impurities. For this reason, the ionization energy (i.e., the energy when thermoelectrons emitted from the filament 23 collide with molecules or atoms) is adjusted to suppress the ionization of Ne. In the present embodiment, 35 eV is used as an example. Since the energy required to ionize Ne is significantly higher than the ionization energy of other atoms and molecules, by adjusting the ionization energy, it is possible to suppress the ionization of only Ne while sufficiently ionizing other atoms and molecules.

In addition, if there is no risk of other components overlapping the mass range of a measurement target (for example, Xe), the second controller 37 may perform the measurement by prioritizing sensitivity over resolution. In more detail, by making the peaks thicker, higher ion intensities can be obtained.

In addition, by suppressing the ionization of Ne (20Ne) and using the isotope 21Ne, the first controller 36 reduces the intensity of the Ne peak used in quantification by several orders of magnitude. In excimer laser gases, the concentrations of Xe, Ar, Kr, and the like in Ne gas must be kept within certain concentration ranges. However, to keep the concentrations within certain ranges, it is necessary to monitor these concentrations with high accuracy. The first controller 36 can reduce the intensity of the Ne peak used for quantification by several orders of magnitude, which makes it possible to bring the ratios of the respective peaks of Ne, Ar, and Xe (as one example, in the case of ArF laser gas) closer together. Likewise, by using the isotope 36Ar for Ar, it is possible to reduce the intensity of the Ar peak used for quantification by several orders of magnitude. By appropriately selecting the isotopes of each component contained in the gas, these peak intensities can be brought closer together, which improves the accuracy of calculations of concentrations.

In addition, by detecting 21Ne and 36Ar, which are isotopes with low abundance ratios, and calculating the concentration of each component, it is not necessary to detect ions with large peak currents, such as 20Ne and 40Ar. As a result, deterioration of the detector 26 can be reduced, making it possible to provide the gas analyzer apparatus 1 that can be used for a long working life.

In addition, the mixture ratio output apparatus 38 can always use the peak intensity of Ne as a reference and calculate concentrations (mixture ratios) from the ratio of the Ne peak intensity to the peak intensities of other components. The concentrations of impurities usually are directly calculated from peak intensities. Here, the peak intensity will change depending on the amount of gas introduced into the mass spectrometer, so when quantification is performed in a system where the pressure of the measurement environment changes, it is necessary to keep the amount of gas introduced into the mass spectrometer constant. With the present method, by calculating concentrations with the Ne peak as a reference, even slight changes in the pressure inside the mass spectrometer can be canceled out.

Note that although an example of a mass filter that uses a quadrupole type as the filter 25 has been described above, the filter 25 may be of another type, such as TOF, an ion trap, or a Wien filter, so long as the filter is capable of selecting, sorting, and turning on and off components containing molecules and/or atoms according to the mass-to-charge ratio m/z.

Note that although specific embodiments of the present invention have been described above, various other embodiments and modifications will be conceivable to those of skill in the art without departing from the scope and spirit of the invention. Such other embodiments and modifications are addressed by the scope of the patent claims given below, and the present invention is defined by the scope of these patent claims.

1. A system comprising an analyzer that analyzes components contained in a gas,
the analyzer including:
a filter that is configured to pass components contained in the gas selectively; and
a detector that is configured to detect components that have passed through the filter, and
the system further comprises:
a first controller that is configured to set the filter so that the detector detects at least one of low-abundance isotopes of a first component as the first component contained in the gas at a first intensity, in place of a high-abundance isotope of the first component;
a second controller that is configured to set the filter so that the detector detects a second component at a second intensity, the second component being contained in a trace amount in the gas, relative to the first component, and
an output apparatus that is configured to acquire a mixing ratio of the second component to the first component from the second intensity and the first intensity without detecting the high-abundance isotope of the first component.

2. The system according to claim 1,
wherein the first controller is configured to set the filter so that only one of the low-abundance isotopes of the first component is detected by the detector at the first intensity.

3. The system according to claim 1,
wherein the second controller is configured to set the filter so that the detector detects the second component contained in the gas at a lower resolution than a resolution for selecting one of the at least one low-abundance isotopes of the first component.

4. The system according to claim 1,
wherein the analyzer further includes an ionizer that is configured to ionize components contained in the gas upstream of the filter, wherein an ionization energy of the ionizer is set to suppress ionization of the first component.

5. The system according to claim 1,
wherein the analyzer further includes:
an ionizer that is configured to ionize components contained in the gas upstream of the filter; and
a third controller that is configured to set an ionization energy of the ionizer to suppress ionization of the first component.

6. The system according to claim 4,
wherein the ionizer includes a filament that is configured to emit thermal electrons.

7. The system according to claim 1,
further comprising a chamber configured to hold the gas upstream of the analyzer temporarily, and
an exhaust system that is configured to control an amount of gas flowing into the chamber to keep a pressure P inside the chamber at a condition indicated below.
0.005 Pa<P<0.05 Pa.

8. The system according to claim 1, wherein the first component includes an inert gas.

9. The system according to claim 1, wherein the filter includes a quadrupole filter.

10. The system according to claim 1,
further comprising:
a processing apparatus into which or from which a process gas flows or is exhausted;
a supply apparatus that is configured to supply at least one of an input gas, an intermediate gas, and an output gas as the process gas of the processing apparatus to the analyzer apparatus; and
a management apparatus that is configured to manage the processing apparatus or a process being performed by the processing apparatus based on analysis results of the analyzer.

11. A control method for a system including an analyzer that analyzes components contained in a gas,
wherein the analyzer includes:
a filter that is configured to pass components contained in the gas selectively; and
a detector that is configured to detect components that have passed through the filter,
wherein the method comprises detecting a first component contained in the gas using the detector with a controller selecting, using the filter, at least one of low-abundance isotopes of the first component at a first intensity, in place of a high-abundance isotope of the first component,
detecting a second component contained in a trace amount in the gas, relative to the first component, using the detector at a second intensity, by selecting using the filter, and
outputting a mixing ratio of the second component to the first component acquired from the second intensity and the first intensity, without detecting the high-abundance isotope of the first component.

12. The method according to claim 11,
wherein the detecting the first component with selecting, using the filter, at least one of low-abundance isotopes of the first component includes detecting the first component with selecting, using the filter, only one of the low-abundance isotopes of the first component at the first intensity.

13. The method according to claim 11,
wherein the detecting the second component contained in the gas using the detector, with the controller selecting, using the filter, at a lower resolution than a resolution for selecting the at least one of the low-abundance isotopes of the first component at the second intensity.

14. The method according to claim 11,
wherein the analyzer further includes an ionizer that is configured to ionize components contained in the gas upstream of the filter, and
the method further comprises controlling, by the controller, to set an ionization energy so as to suppress ionization of the first component.

15. The method according to claim 11,
wherein the system further includes:
a chamber that is configured to hold the gas upstream of the analyzer temporarily, and
an exhaust system that is configured to control an amount of gas flowing into the chamber,
wherein the method further comprises controlling, by the controller, to keep a pressure P inside the chamber at a condition indicated below.
0.005 Pa<P<0.05 Pa.

16. The method according to claim 11,
wherein the first component includes an inert gas.

17. The method according to claim 11,
wherein the system further includes:
a processing apparatus into which or from which a process gas flows or is exhausted; and
a supply apparatus that is configured to supply at least one of an input gas, an intermediate gas, and an output gas as the process gas of the processing apparatus to the analyzer apparatus, and
the method further comprises managing, by a management apparatus of the system, the processing apparatus or a process being performed by the processing apparatus based on analysis results of the analyzer.

18. A method for analyzing components contained in a gas using an analyzer,
the analyzer including:
a filter that is configured to pass components contained in the gas selectively; and
a detector that is configured to detect components that have passed through the filter,
wherein the method comprises detecting a first component contained in the gas using the detector with selecting, using the filter, at least one of low-abundance isotopes of the first component at a first intensity, in place of a high-abundance isotope of the first component,
detecting a second component contained in a trace amount in the gas, relative to the first component, using the detector at a second intensity, by selecting using the filter, and
outputting a mixing ratio of the second component to the first component acquired from the second intensity and the first intensity, without detecting the high-abundance isotope of the first component.

19. A control program of the system to perform the control method at the system according to claim 11
comprising an instruction that causes the detecting the first component, the detecting the second component and the outputting the mixing ratio.
